(19) United States
(12) Patent Application Publication
(10) Pub. No.: US 2024/0016690 A1
(43) Pub. Date: Jan. 18, 2024

(54) SUPPORT APPARATUS

(71) Applicant: SPANSET INTER AG, Wollerau Schwyz (CH)

(72) Inventor: David EHNIMB, Thalwill (CH)

(73) Assignee: SPANSET INTER AG, Wollerau Schwyz (CH)

(21) Appl. No.: 18/032,081

(22) PCT Filed: Oct. 15, 2021
(86) PCT No.: PCT/EP2021/078631

§ 371 Date: Apr. 14, 2023

(30) Foreign Application Priority Data
Oct. 15, 2020 (DE) 10 2020 006 331.4

(51) Int. Cl. A61H 3/00

(52) U.S. Cl. CPC A61H 3/00; A61H 2201/0214; A61H 2201/165

(57) ABSTRACT

The invention relates to an apparatus for supporting human movements or for protecting against falls, wherein the apparatus can be arranged on a body of a person, the apparatus has contact surfaces with the body, and the contact surfaces are formed completely or partially by at least one contact element, the at least one contact element consists completely or partially of a PCM and the extent to which the at least one contact element bears on the body can be adjusted, and wherein the at least one contact element (2) on the side facing the body of the person (6) has a smaller bearing surface in the resting state than in the movement state.

The invention relates to an apparatus for supporting human movements, so-called exoskeletons, or for protecting against falls, wherein the apparatus can be arranged on a body of a person, the apparatus has contact surfaces with the body and contact elements are provided/arranged on the contact surfaces, which contact elements consist completely or partially of a phase change material (PCM).

Apparatuses for supporting human movements (exoskeleton) or for protecting against falls have been known for a long time. It is particularly important for the products to be comfortable to wear. Wearing such apparatuses means that the body of the person receives an extra cover. In an active state, also referred to as a movement state, it is more difficult for thermal energy to dissipate from the body. The person begins to sweat and finds the thermal energy unpleasant.

In principle, the use of PCMs in prostheses and orthoses and also exoskeletons is known, for example from DE 10 2014 011 373 A1.

In this case, the phase change material has the property that it melts or evaporates at body temperature of a person. For this process it requires thermal energy, which is provided by the body temperature of the person, as described above in the movement state of the person. During the melting or evaporating process, a cooling effect can be felt at contact surfaces of the apparatus.

However, this effect is not available indefinitely. The object of the invention is to provide an apparatus for supporting human movements or for protecting against falls, which is optimised compared to previous apparatuses.

This object is achieved according to the invention by an apparatus having the features of the independent claim 1, wherein the subclaims contain further embodiment variations of these solutions according to the invention.

Thus an apparatus for protecting a person from falling or for supporting human movement, a so-called exoskeleton, where the apparatus can be arranged on the body of a person, has the following features:

The apparatus has contact surfaces with the body. The contact surfaces are formed completely or partially by at least one contact element. The at least one contact element consists completely or partially of a phase change material (PCM), wherein the extent to which the at least one contact element bears on the body of the person can be adjusted. In this respect, the bearing surface of the contact element can be adjusted. It should be noted that within the meaning of the invention contact surfaces can also be only one contact surface. The wording has only been chosen for the sake of clarity in this description.

This makes it possible to extend the period of the phase change of the PCM or even to control it as necessary so that a cooling effect can be optimised when using the apparatus.

Furthermore, it can be provided that the extent to which the least one contact element bears on the body can be adjusted as a function of a resting state and a movement state of the person or the apparatus for supporting human movement, i.e. the exoskeleton. Particularly in the case of protection from falls, this depends on the movement state of the person and in the case of exoskeletons it can also depend on the state of the apparatus itself, i.e. whether this is activated or not.

In the resting state of the apparatus or person, the at least one contact element on the side facing the body of the person can then have a smaller bearing surface and the apparatus can thus have a smaller contact surface than in the movement state. This results in a delayed phase change of the PCM in the resting state. The apparatus then, for example, rests less tightly on the body or contact elements have a shape that allows for a bearing surface, for example a counter form to the body shape or body part shape.

It can also be provided that at least one contact element on the side facing the body of the person in the resting state of the apparatus or of the person has elevations and depressions, i.e. an uneven surface. This has the advantage that there is also a delayed phase change in the resting state in this case, because the contact surface is increased in the resting state compared to the movement state. In the movement state the apparatus then has no or fewer elevations and depressions on the side facing the body of the person. This then has the advantage of an increased cooling effect in the movement state.

Furthermore, it can be provided that the extent to which the at least one contact element bears on the body is regulated by applying pressure to the at least one contact element. In this way the elevations and depressions or the extent to which the at least one contact element bears on the body of the person can be regulated. The pressure can be applied by any suitable means known to the person skilled in the art.

In this respect, the extent to which the at least one contact element bears on the body and thus also the pressurisation of the at least one contact element can be regulated pneumatically, preferably by means of at least one volume-adjustable air chamber. Deforming the at least one air chamber as a result of pneumatic pressurisation also has an effect on the at least one contact element, which thus changes the extent to which it bears on the body or the flatness of the side of the contact element facing the body of the person.

The at least one contact element is thus in active connection with the at least one air chamber. The at least one contact element is underpinned for example by the at least one air chamber or contact elements alternate with the at least one air chamber. It is thus possible to easily control the contact surface of the contact elements via the expansion of the air chambers. However, the specific arrangement of the at least one air chamber as such is not predetermined. This depends on the respective structure of the apparatus.

Furthermore, the volume of the at least one air chamber, i.e. the pneumatic pressurisation, can be regulated by means of a pump, for example a hand pump or a mechanical pump and a valve control. Mechanical pumps are all known pumps which are not hand operated. The pumps can be attached directly to the apparatus or external pumps can be used. The valve control can be arranged for example on the pump itself or on the at least one air chamber or on lines between the air chamber and pump, as a discharge valve for example, in order to reduce a pneumatic pressurisation again by means of the pump. Valve control within the meaning of the invention includes all pneumatic controls which the person skilled in the art considers suitable for the specific case of providing pressure regulation in addition to the pump.

A further way of regulating the volume of the at least one air chamber is that the at least one air chamber is pressurised by at least one further air chamber which is connected to the at least one air chamber, wherein the at least one further air chamber reduces its volume due to a movement of the person when using the apparatus and thus the at least one air chamber is pressurised. There are thus corresponding air chambers which change their volume depending on the movement of the person and thus also the apparatus.

It is also possible that the at least one contact element is part of a support and/or application device of the apparatus for body parts of the person, for example a head rest. If at least one contact element is attached to a headrest, then the person using the apparatus can rest and cool their head during a resting phase.

As already shown, the apparatus described above makes it possible to extend the period of the phase change of the PCM or even to control it as necessary so that cooling can be optimised when using the apparatus.

The invention is explained further in the following by way of an exemplary embodiment with reference to the Figures. Further advantages, features and embodiments of the invention are described.

In the Figures:

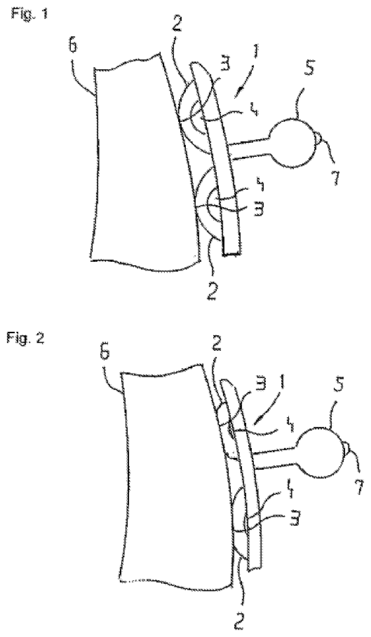

FIG. 1 shows a schematic representation of an apparatus in the resting state,

FIG. 2 shows a schematic representation of an apparatus in the movement state.

FIG. 1 and FIG. 2 show a portion of an apparatus 1 according to the invention in the form of an exoskeleton. The apparatus 1 is arranged on a body of a person 6 and has contact surfaces 3 in contact with the body of the person 6. The contact surfaces are formed by two contact elements 2, which consist of a PCM (phase change material) or contain the latter. There is a known structure for a contact element made of PCM in the form of cushion.

In the exemplary embodiment the extent of to which the two contact elements bear on the body is regulated by pressurising the contact elements 2. This is ensured pneumatically in the exemplary embodiment by means of two air chambers 4 with adjustable volumes, each of which underpin the two contact elements 2. Thus the respective air chamber 4 is in active connection with the respective contact element 2. The volume of the two air chambers 4 and thus the pneumatic pressurisation is regulated in the exemplary embodiment by means of a pump 5 with valve control 7, in the exemplary embodiment a hand pump, which is arranged on the apparatus 1 and is easily accessible. The valve control 7 in the exemplary embodiment is a discharge valve, which can be activated via a push button.

Depending on the pneumatic pressurisation of the two air chambers 4 by means of the pump 5 and the valve control 7, the extent to which the two contact elements 2 bear on the body of the person 6 can be adjusted. In this respect, the bearing surface of the contact element can be varied and thereby the contact surface 2 of the apparatus 1.

This ensures that the two contact elements on the side facing the body of the person 6 have a smaller bearing surface in the resting state, FIG. 1, than in the movement state, FIG. 2. This makes it possible to extend the phase change period of the phase change material (PCM) contained in the contact elements and to control it as necessary by means of the hand pump with valve control, so that cooling can be optimised when using the apparatus.

### LIST OF REFERENCE SIGNS

1. apparatus

2. contact elements

3. contact surface

4. air chamber

5. pump

6. person (section)

7. valve control

In light of the aforementioned overall disclosure, the present invention further comprises the embodiments numbered below:

1. Apparatus (1) for supporting human movements or for protecting against falls, wherein the apparatus (1) can be arranged on a body of a person (6), the apparatus (1) has contact surfaces (3) with the body and the contact surfaces (3) are formed completely or partially by at least one contact element (2), the at least one contact element (2) consists completely or partially of a PCM and the extent to which the at least one contact element (2) bears on the body can be adjusted.
2. Apparatus (1) according to Embodiment 1, wherein the at least one contact element (2) can be adjusted in the extent to which it bears on the body as a function of a resting and a movement state of the person (6) or the apparatus (1) for supporting human movement.
3. Apparatus (1) according to Embodiment 1 or 2, wherein the at least one contact element (2) on the side facing the body of the person (6) has a smaller bearing surface than in the movement state.
4. Apparatus (1) according to Embodiment 1 or 2, wherein the at least one contact element (2) has elevations and depressions on the side facing the body of the person (6) in the resting state and has no or fewer elevations and depressions on the side facing the body of the person (6) in the movement state.
5. Apparatus (1) according to any one of Embodiments 1 to 4, wherein the extent to which the at least one contact element (2) bears on the body is regulated by pressurising the at least one contact element (2).
6. Apparatus (1) according to any one of Embodiments 1 to 5, wherein the extent to which the at least one contact element (2) bears on the body is regulated pneumatically, preferably by means of at least one volume-adjustable air chamber (4).
7. Apparatus (1) according to embodiment 6, wherein the at least one contact element (2) is in active connection with the at least one air chamber (4).
8. Apparatus (1) according to any one of Embodiments 6 or 7, wherein the volume of the at least one air chamber (4) is regulated by means of a pump (5) and a valve control (7).
9. Apparatus (1) according to any one of Embodiments 6 to 8,wherein a pressurisation of the at least one air chamber (4) is performed by at least one further air chamber, which is connected to the at least one air chamber (4), wherein the at least one further air chamber is reduced in volume by a movement of the person (6) when using the apparatus (1) and thus the at least one air chamber (4) is pressurised.
10. Apparatus (1) according to any one of Embodiments 1 to 9, wherein the at least one contact element (2) is part of a support and/or application device of the apparatus (1) for body parts of the person (6).

1. An apparatus (1) for supporting human movements or for protecting against falls, wherein the apparatus (1) can be arranged on a body of a person (6), the apparatus (1) comprising:
contact surfaces (3) with the body and the contact surfaces (3) are formed completely or partially by at least one contact element (2), the at least one contact element (2) consists completely or partially of a PCM and the extent to which the at least one contact element (2) bears on the body can be adjusted, and wherein the at least one contact element (2) on the side facing the body of the person (6) has a smaller bearing surface in the resting state than in the movement state.

2. The apparatus (1) according to claim 1, wherein the at least one contact element (2) can be adjusted in the extent to which it bears on the body as a function of a resting and a movement state of the person (6) or the apparatus (1) for supporting human movement.

3. The apparatus (1) according to claim 1, wherein the at least one contact element (2) has elevations and depressions on the side facing the body of the person (6) in the resting state and has no or fewer elevations and depressions on the side facing the body of the person (6) in the movement state.

4. The apparatus (1) according to claim 1, wherein the extent to which the at least one contact element (2) bears on the body is regulated by pressurizing the at least one contact element (2).

5. The apparatus (1) according to claim 1, wherein the extent to which the least one contact element (2) bears on the body is regulated pneumatically, preferably by means of at least one volume-adjustable air chamber (4).

6. The apparatus (1) according to claim 5, wherein the at least one contact element (2) is in active connection with the at least one air chamber (4).

7. The apparatus (1) according to claim 5, wherein the volume of the at least one air chamber (4) is regulated by means of a pump (5) and a valve control (7).

8. The apparatus (1) according to claim 5, wherein a pressurization of the at least one air chamber (4) is performed by at least one further air chamber, which is connected to the at least one air chamber (4), wherein the at least one further air chamber is reduced in volume by a movement of the person (6) when using the apparatus (1) and thus the at least one air chamber (4) is pressurized.

9. The apparatus (1) according to claim 1, wherein the at least one contact element (2) is part of a support and/or application device of the apparatus (1) for body parts of the person (6).
